(19) United States
(12) Patent Application Publication
(10) Pub. No.: US 2024/0197800 A1
(43) Pub. Date: Jun. 20, 2024

(54) Aristotelia chilensis EXTRACTS FOR TREATING FUNGAL INFECTIONS

(71) Applicants: Phytonet Doo Beograd-rakovica, Belgrade (RS); Institut Za Molekularnu Genetiku I Geneticko Inzenjerstvo Univerziteta E Beolgrade, Belgrade (RS)

(72) Inventors: Bojan Pavlovic, Belgrade (RS); Katarina Savic Joncic, Belgrade (RS); Cem Aydogan, Schindellegi - Feusisberg (CH); Lidija Senerovic, Belgrade (RS); Lidija Djokic, Belgrade (RS); Ivana Moric, Belgrade (RS); Aleksandar Pavic, Belgrade (RS); Nada Stankovic, Belgrade (RS)

(21) Appl. No.: 18/286,095

(22) PCT Filed: Apr. 6, 2022
(86) PCT No.: PCT/RS2022/000005

§ 371 Date: Oct. 6, 2023

(30) Foreign Application Priority Data
Apr. 7, 2021 (RS) P-2021/0431

(51) Int. Cl. A61K 36/185; A61K 31/352; A61P 31/10

(52) U.S. Cl. CPC A61K 36/185; A61K 31/352; A61P 31/10

(57) ABSTRACT

This invention provides for a prophylactic and therapeutic natural and safe agent, which contains Aristotelia chilensis berry extract as an active substance or extracts of other berries, for the prevention and treatment of fungal candidiasis infections in the digestive, urogenital tract but also in systemic diseases caused by fungi of the genus Candida especially Candida albicans, by the mechanism of filamentation inhibition. Other berries can be from the group of chokeberry, cranberry, blueberry, currant or other berry fruit.

### TECHNICAL FIELD

This invention belongs to the field of pharmaceutical products and is related to the use of a natural preparation, an extract of plant berries with a high content of anthocyanin delphinidin, from fruits of plants such as Aristotelia chilensis, known as Maqui berry, for treating or for prophylaxis of fungal infection caused by fungi of the genus Candida, especially Candida albicans. According to the IPC, the invention belongs to classes A61K36/00 and A61P31/10.

### TECHNICAL PROBLEM

Fungi belong to a group of microorganisms that are part of the normal human microbiome and they inhabit the mucous membranes, skin, genitals, as well as digestive tract, where they assist food absorption and digestion. One of the most well-known species of fungi present in humans is Candida (Candida albicans) from the Ascomycetes group. Its excessive reproduction due to various factors causes candidiasis—one of the most common fungal infections of the oral cavity, digestive tract, vagina, and other mucous membranes in the body and even the skin. Candidiasis on the affected areas leads to inflammation and causes itching, tingling, and edema. Candida can reappear after a seemingly successful antifungal treatment suppressed the infection, and can progress into a chronic infection. If the human immune system is not damaged and functions with its full potential, this type of infection rarely develops into a more serious pathological condition. However, if the immune system is compromised, candida migrates from the primary infection site, and it spreads to internal organs, which seriously, even vitally, endangers the infected person.

In treating candidiasis, there are following technical problems that cannot be solved by well-known therapeutics:
Candida is difficult to treat,
Candida easily and quickly becomes resistant to antimycotics,
known drugs are toxic,
known drugs are insufficiently effective,
Candida returns or cannot be eliminated from the body.

The known antimycotics either stop the growth of candida or kill it, thus forcing it to create resistance.

Due to the above-mentioned, there is a need for an effective and harmless natural preparation that can be used both preventively and in the treatment of persistent chronic fungal infections caused by the growth of yeasts of the genus Candida.

### BACKGROUND ART

Polyphenols are a large group of bioactive compounds found in plants and plant foods. It is known that among polyphenols, there is a large group of compounds belonging to flavonoids, called anthocyanins, which modulate various biochemical/signaling pathways, thus participating in the promotion of beneficial properties of herbal preparations, including vasoprotective effects, improving cognitive abilities, and muscle performance. They are also known to have anticancer activities. Anthocyanins, glycosides of anthocyanidins, are responsible for colours that are widely distributed in fruit berries. These natural non-toxic pigments are water-soluble compounds and are divided into 6 classes: malvidin, elphinidin, petunidin, pelargonidine, cyaniding, and peonidin. The position and number of hydroxyl and methyl groups in the basic structure are crucial for their ability to remove free radicals.

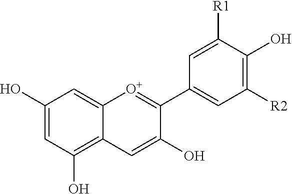

| R1 | R2 | anthocyanidin |
|---|---|---|
| OH | OH | delphinidin |
| OH | H | cyanidin |
| H | H | pelargonidin |
| OCH₃ | H | peonidin |
| OCH₃ | OH | petunidin |
| OCH₃ | OCH₃ | malvidin |

Delphinidin is an anthocyanidin, a primary plant pigment, known as a very strong antioxidant from plants of the genus Delphinium, which has about 300 species, perennial flowering plants in the Ranunculaceae family, which can be found throughout the northern hemisphere, but also on high mountains of tropical Africa.

Delphinidin is blue and gives colour to plant flowers from the genus Viola and Delphinium. It also gives a bluish red colour to grapes used in the production of Cabernet Sauvignon, and can be found in cranberries and Concord grapes, blueberries, as in other berries. Delphinidin, like most other anthocyanidins, is sensitive to pH, i.e. it is a natural pH indicator, which changes colour to red in an acidic environment, and to blue in a basic environment.

Several glycosides derived from Delphinidin are known, and they are:

Myrtillin (delphinidin-3-O-glucoside) and tulipanin (delphinidin-3-O-rutinoside) found in blackcurrant pomace,

Violdelphin (Delphinidin 3-rutinoside-7-O -(6-O-(4-(6-O-(4-hydroxybenzoyl)-b-D-glucosyl)-oxybenzoyl)-b-D-glucoside) which is responsible for the light purple—the hue of the flower Aconitum chinense and

Nasunin (Delphinidin-3-(p-coumaroyl rutinoside)-5-glucoside) which gives color to the purple skin of the eggplant.

There are many types of berries whose fruits are rich in anthocyanins and their glycosides and delphinidin. Nowadays, it is already reliably known that one of such plants is Aristotelia chilensis (Maqui berry) and that its fruits are the richest in delphinidin. It is a very resistant plant, also known as Maqui or Chilean grapevine. Aristotelia chilensis is a species of tree in the family Elaeocarpaceae native to South America and inhabits the temperate rainforests of Chile and neighboring regions of southern Argentina. This type of fruit that is sold on the market mainly comes from the wilderness, and it is organic, which is a great advantage over other cultivated berries.

Among the natives of Chile, it has long been used as a traditional medicine in the treatment of diarrhea, inflammation, and fever. Japanese scientists were the first to isolate the active components, showing that it is very effective in treating dry eye syndrome. There are numerous papers that show different activities of Aristotelia chilensis fruit extract in treating various eye diseases, but also in the regulation of blood glucose levels.

Small purplish-black Maqui berries taste similar to blackberries, and studies of the qualitative composition of polyphenols have shown that their anthocyanins contain eight glucoside pigments of Delphinidin and cyanidin, with the main anthocyanin Delphinidin 3-sambubiozid-5-glucoside (34% of total anthocyanins).

Maqui berries, as a representative of berries rich in anthocyanins, are known to have numerous advantages over other similar berries. In addition to being an excellent antioxidant, Maqui berries are also rich in polyphenols, anthocyanins, and other active ingredients, which help neutralize free radicals, which are the main responsible agents that cause aging. As the strongest natural antioxidant, it neutralizes free radicals, detoxifies, protects the skin from harmful effects of sunlight, strengthens bones and joints, improves the immune system, improves energy levels, and reduces the oxidation of LDL cholesterol.

According to the National Food and Technology Institute of Chile—INTA, this rich soil is a unique bioclimatic area with aggressive climatic conditions where vegetation thrives and is located between the Andes and cold Humboldt currents with high levels of solar radiation and large temperature deviations. Plants growing in these conditions contain unique phytochemical profiles that have not been found anywhere else on the planet. These plants contain 30-40% more bioactive molecules than similar ones grown in other countries, such as chokeberry, blueberry, black currant. The climate in this area improves the quality of fruits, resulting in a higher percentage of molecules useful for human health, and indigenous fruits are carefully selected and picked by families of pickers from wild forests and fields, following old traditions preserved for centuries.

According to the US Department of Agriculture ORAC data from May 2010, Maqui berry is the strongest natural antioxidant, 7 times stronger than acai berry, and 9 times stronger than goji berry. Its anthocyanin composition is about 25% cyanidin and up to 75% delphinidin. Both anthocyanins and delphinidin have been extensively investigated and their multiple health benefits have been proven. The predominance of Maqui berry activity over other related plants and berries is caused by the fact that it is the richest in delphinidin.

According to a USDA source from September 2015, Maqui berries contain 1,260.00 mg of delphinidin per 100 g of berries, while the closest behind it is Blueberry (Vaccinum myrtilus) with 97.59 mg.

Maqui berry is, therefore, considered to be the most powerful antioxidant superfruit, and it is also called berry superfood.

Clinical trials have shown both preventive and curative effects of Aristotelia chilensis fruit extract in inflammatory diseases, oxidative stress, as well as metabolic diseases. Due to its various beneficial effects on overall human health, Aristotelia chilensis fruit extract was selected to test the activity in this study, and due to the fact that it is standardized and has the highest content of delphinidin as the most active anthocyanin, the strongest antioxidant effect of all other similar extracts.

By browsing through available patent and scientific literature on pharmaceuticals for the treatment and prevention of candidiasis and other fungal infections based on active principles of natural origin such as plant extracts of berries, including Maqui berry—Aristotelia chilensis, containing anthocyanins known as delphinidin, the following was found:

Patent EP2344154 describes combinations of anthocyanidins rich in delphinidin, including delphinidin found in berries, among others and Aristotelie chilensis. However, the described compositions contain additional andrographolides, such as those found in plants of the genus Andrographis, or compositions containing combinations of myrtillin, quercetin or caffeoyl quinic derivatives and proanthocyanidins, such as those found in plants of the genus Vaccinium.

Patent RS59259 B1, EP3209315 B1 describes a preparation related to combinations containing at least one compound of molecules consisting of at least: one Chrysanthellum Indicum extract and one Sunaga Scolymus extract and one Vaccinum myrtillus extract and synthetic piperine and/or pepper extract containing piperine, wherein such a compound molecule comprises: at least 1 of the following molecules: apigenin-7-O-glucuronide, chrysanthemum A, chrysanthemum B, caffeic acid, luteolin, maritimein, eriodictyol, isocanin, apigenin-8-α-L-arabinoside-6-C-β-D-glucoside (shaftoside), apigenin-6,8-C-di-β-D-glucopyranoside (vicenin-2), and at least 1 of the following molecules: monocaffeoyl acid, delphinidin-3-galactoside, Delphinidin -3-glucoside, cyanidin-3-galactoside, Delphinidin-3-arabinoside, cyanidin-3-glucoside, petunidin-3-galactoside, cyanidin-3-arabinoside, petunidin-3-glucoside, peonidin-3-galactoside, peonidin-3-galactoside, petunidin-3-arabinoside, peonidin-3-glucoside, peonidin-3-glucoside, malvidin, malvidin-3-glucoside, malvidin-3-arabinoside i-piperine. The subject combinations are not the subject of the patent solution described herein. This invention is intended to regulate the metabolism of carbohydrates and fats in humans and animals.

WO2017113032A1 application discloses an invention related to the natural antibacterial and antioxidant composition of an extract rich in phenolic compounds obtained from A. chilensis seedlings cultured in vitro. In particular, the described method is for the creation of plant biomass in a sustainable way, without the need to use materials grown in natural conditions, which reduces the impact on the exploitation of the species. This patent is not contrary to the present invention.

Patent JP2013107825B describes an antioxidant composition containing Maqui berry.

Patent TW201709920A application describes a composition containing Maqui berry and more fruits and berries with antioxidant capacity.

Patent US2014377338A1 application describes a Maqui berry extract containing anthocyanins that have a very high oxygen radical absorption capacity (ORAC), and relates to topical application in the prevention of skin aging.

Patent RO131883 describes the antifungal activity of chitosan isolated from A. chilensis.

Patent US20050158396 application describes antioxidant properties of plant anthocyanosides and their use in cosmetics.

However, the closest state of the art is found in three scientific papers:
1) The paper entitled “Antioxidants and antimicrobial extracts of Aristotelia chilensis and Ugni molinae and its applications as preservatives in cosmetic products”, Marcia AVELLO1 * et al., describes the effects of extracts of Aristotelia chilensis and Ugni molinae leaves on Candida albicans.
2) The paper entitled “The powerful colour of the Maqui” by Carolina Fredesa, * and Paz Robertb, describes the performance of anthocyanins as antioxidants with anti-inflammatory and anti-diabetic effects.
3) The paper entitled “Effects of magnolol and honokiol on adhesion, yeast-hyphal transition, and formation of biofilm by Candida albicans (2015) PlosOne, 0 (2): e0117695, Lingmei Sun et al. describes the effects of magnolol and honokiol, natural products isolated from the bark of the magnolia tree (Magnolia officinalis) in preventing candida virulence.

From the above-mentioned, it is evident that in the literature and state of the art there are no papers or patents related to the use of berry extracts of plants including Aristotelia chilensis on Candida albicans, or the use of extracts with high content of delphinidin (as one of the anthocyanins present in this fruit) in the treatment of candidiasis.

Moreover, from the above state of the art, it is known that the fruit extract of Aristotelia chilensis (Maqui berry) has antioxidant and anti-inflammatory effects, and that it is used to treat diabetes, eye diseases, for cosmetic purposes, for hair care, skincare, for treating atopic dermatitis, skin whitening, rosacea, as painkillers, for treating menstrual syndrome, as a dietary supplement, but also in the treatment of hepatitis C, leukemia and cancer. No papers have been found on treating candidiasis or other fungal infections using extracts of this or other berries.

### THE DESCRIPTION OF THE INVENTION

Candida albicans and other species of the genus Candida (e.g. C. glabrata, C. krusei, C. tropicalis)—collectively referred to as Candida species, are fungi that survive best on warm and humid surfaces, and can usually be found on the skin and mucous membranes of organs for digestion and reproduction, where they live as part of the normal flora. The most common cause of fungal infections (mycosis) in humans is Candida albicans, which naturally inhabits the surface of the intestinal fold, in smaller numbers, and which under various influences can rapidly and unnaturally multiply, and lead to pathological conditions. The most common factors that affect the excessive development of candida in the body are the use of antibiotics, oral contraceptives, hormone therapy, diabetes, as well as weakened immunity, or the use of immunosuppressive drugs.

C. albicans is part of the normal physiological flora in 80% of the human population, where it does not cause harmful effects, while the excessive growth of this fungus and reproduction are manifested by the appearance of candidiasis. Candidiasis can affect the oral cavity, digestive tract, genital tract, and is not uncommon in diabetics as well as in pregnancy. Candidiasis is actually a disorder of the normal flora.

There are many reasons that lead to the excessive growth of this fungus. These include a diet rich in sugar, processed carbohydrates, and the use of alcohol, which allows this yeast to multiply. An improper lifestyle impairs the health of the oral, intestinal, and vaginal flora, which is an ideal condition for the reproduction of candida. Smoking, stress, alcohol, sugar, consuming yeast dough, and many other daily habits change the pH value in our body, which leads to the creation of conditions for excessive reproduction of candida.

In case of severe damage of the immune system, which is manifested by a drastic drop in the number of leukocytes—in oncologic, hematologic, and HIV patients, or in case of the immaturity of the immune system as in premature infants, fungi can penetrate the bloodstream and internal organs.

Broad-spectrum antibiotics are used to treat bacterial infections. After a long-term use or repeated use of several antibiotics, the body can become a breeding ground for fungi. This risk is increased in women who use oral contraceptives.

Asthma therapy using inhaled corticosteroids increases the risk of developing this fungal infection in the mouth and its rapid transition to a systemic spread of candida.

In patients receiving radiation therapy or chemotherapy, as many as one-third of patients develop an invasive candida infection at some point. The reason for this is that both chemotherapy and radiation, in addition to killing cancer cells, also kill good bacteria and thus enable the growth of candida. People with type 1 and type 2 diabetes have high levels of sugar in their mouths. Candida is a yeast that feeds on sugar, and diabetics are people at an increased risk of developing this fungal infection.

Any person with a weakened or compromised immune system has an increased risk of developing this fungal infection, including infants, children, the elderly, cancer patients, and people living with HIV, as well as people with autoimmune diseases.

Drugs of choice for local infections are agents that act locally, directly on the infected area. The development of antifungal resistance in fungi depends on the type of drug used for treatment and it is very common in treatments based on azoles such as miconazole, clotrimazole, fluconazole and itraconazole, which are most commonly used in the treatment of oropharyngeal candidiasis. Resistance mainly occurs in immunocompromised individuals, because they receive a long-term preventive antifungal therapy. One of the negative effects of clinically approved antifungals, in addition to their toxicity, is the killing of fungi, which further disrupts the normal flora.

Proper nutrition and probiotic cultures can be a solution for mild forms of candidiasis, but the basic problem with these conditions is the recurrence of Candida infection that is suppressed in the pockets of the digestive tract or oral cavity every time the conditions are met.

This invention is an unexpectedly discovered new biological activity of natural products obtained from fruit extraction, of berry fruits containing anthocyanins, which are most abundant in Chilean Maqui, Aristotelia chilensis, called Maqui Berry, and which are rich in vitamins, minerals and anthocyanins, among which the most active and most frequent is delphinidin, as well as other medicinal alkaloids, which are antioxidants. This new biological activity is reflected in prevention of filamentation of fungi of the genus Candida. Filamentation is a morphogenetic process during which the candida changes from a non-pathogenic (yeast) form to an invasive, pathogenic (filamentous) form, which is very dangerous for the integrity of epithelial tissues and on which the cells of the immune system have a weak effect. Preventing filamentation reduces virulence and makes candida an easy target for antimycotics and immune system cells.

The standardized extract of Aristotelia chilensis was used in the study of biological activity, because the content of anthocyanins in these berries is 3 to 4 times higher than in blueberries, cranberries, or other berries, and this extract has an effective antioxidant effect and can be used. as a therapeutic, but also preventively as a dietary supplement. This invention represents an unexpected discovery that the tested extract of the fruit Aristotelia chilensis inhibits the process of filamentation of fungi of the genus Candida. The observed surprising effect in this study is the fact that the tested extract of Aristotelia chilensis does not behave fungicidally, i.e. it does not destroy Candida, which would disrupt the normal microflora, but prevents its transition from non-pathogenic into pathogenic form.

### The Best Way to Apply the Invention

The extract, which is the subject of this invention, and its ingredients have shown to be effective in preventing and/or treating infections caused by fungi of the genus Candida. The invention relates to the use in the prevention and treatment of infections caused by fungi of the genus Candida, especially Candida albicans, using natural preparations isolated from berries of the plant Aristotelia chilensis, but also similar plants in terms of the composition of the fruit extract.

From the above-mentioned, it is evident that there is a need to find a safe natural preparation that would have a long-term impact on eliminating the risk of reproduction of this fungus, which is currently an unresolved problem. Drugs in clinical use are effective immediately, and a new growth of fungi occurs as soon as some of the above factors that are suitable for reproduction are acquired, because Candida, when treated with clinical antimycotics, withdraws to hidden parts of the tract, where it remains active and inaccessible to therapeutics.

This patent application reveals the discovery of the use of a natural and safe preparation that has no fungicidal effect but prevents the filamentation of naturally occurring Candida, which prevents its transition to an aggressive and pathogenic form.

In the process of testing the biological activity of berry fruit extracts, despite the fact that all types of this fruit contain similar active substances, Aristotelia chilensis extract was used due to its standardized and high content of delphinidin as the strongest antioxidant. The results of the experimental research are applicable to all plant extracts containing cyanidin and delphinidin. During the testing of Aristotelia chilensis fruit extract on different models for testing biological activities, this invention unexpectedly revealed that on one of the models, the zebrafish model (Danio rerio, zebrafish,), the formation of Candida hyphae lacked, which is a laboratory method for monitoring its filamentation in vivo and transition to a virulent, that is pathogenic form.

The biomedical application of plant extracts and their purified ingredients mainly depends on the range between the minimum effective (therapeutic) dose and the minimum toxic dose. For such an assessment, it is important to choose a suitable model that would evaluate both very important effects, for use in therapeutic purposes.

The zebrafish model has been proven in the extensive scientific literature as a very reliable experimental animal model for detecting biological activities and assessing the toxicity of plant extracts and other natural products. Such reliability is possible thanks to the high molecular-genetic, physiological and immune similarity between zebrafish and humans, as well as the high degree of correlation between them in response to drugs.

During the study of bioactivity and toxicity of the standardized extract of the plant Aristotelia chilensis, which was chosen as one of the sources of anthocyanins—delphinidin, which are present in berries and plants of the genus Ranunculaceae, we used the zebrafish model to assess the biological safety (toxicity) of the extract with the basic goal of discovering all the possibilities for its medical application.

All experiments performed on the zebrafish model were performed exclusively on embryos up to 120 h old, in accordance with the recommendations of European Directive 2010/63/EU and ethical guidelines of the Guide for the Care and Use of Laboratory Animals of the Institute of Molecular Genetics and Genetic Engineering, University of Belgrade (Belgrade, Serbia). During the laboratory testing in order to assess the toxicity and bioactivity of the extract of standardized content of anthocyanins, delphinidin—extract of Aristotelia chilensis, standard tests were performed on an experimental model of zebrafish and the expected results were obtained, i.e. the safety of these extracts was confirmed. Quite unexpectedly, tests showed that the tested extract inhibits the filamentation, reproduction, and spreading of Candida albicans. The invention will be further illustrated by exemplary embodiments without intending to be limited thereto.

### EXAMPLE 1—THE ASSESSMENT OF LETHAL AND TERATOGENIC POTENTIAL

The acute toxicity analysis was performed on zebrafish embryos—FET (Fish Embryo Toxicity assay). The impact of Aristotelia chilensis berry extract on the survival and development of zebrafish embryos was examined according to the OECD 2013 guidelines for testing chemicals and previously described protocols. In the phase of 6 hours after fertilization (hpf), zebrafish embryos were exposed to nine concentrations of aqueous solution of Aristotelia chilensis berry extract, as follows: 10; 25; 50; 100; 250; 500; 1000; 2000 and 2500 μg/mL (corresponding to delphinidin concentrations of minimum 2.5; 6.25; 12.5; 25; 62.5; 125; 250; 250; 500 and 625 μg/mL, and concentrations of minimum 3.5; 8.75; 17.5; 35; 87.5; 175; 350; 700 and 875 μg/mL total anthocyanins). The embryos were exposed to given concentrations in the period up to 120 hpf, and the survival and occurrence of teratogenic malformations were examined every 24 hours. Distilled water was used as a negative control. The experiments were performed three times using 30 embryos per concentration. Different endpoints were used to assess toxicity (Table 1) at 24; 48; 72; 96 and 120 hpf, followed by the LC₅₀ value (concentration that kills 50% of the embryos) and the EC50 value (concentration affecting 50% of embryos), as standard toxicological parameters, were determined.

TABLE 1
Lethal and teratogenic effects monitored in zebrafish embryos
(Danio rerio) at different times after fertilization (hpf).

| | | Exposure time | | | |
|---|---|---|---|---|---|
| Category | Developmental endpoints | 24 | 48 | 72 | 96/120 |
| Lethal effect | Coagulated eggs | ● | ● | ● | ● |
| | Lack of somite formation | ● | ● | ● | ● |
| | Non-detachment of the tail | ● | ● | ● | ● |
| | Lack of the heart beating | ● | ● | ● | ● |
| Teratogenic | Malformation of head | ● | ● | ● | ● |
| effect | Malformation of eyes | ● | ● | ● | ● |
| | Malformation of sacculi/otoliths | ● | ● | ● | ● |
| | Malformation of chorda | ● | ● | ● | ● |
| | Malformation of tail | ● | ● | ● | ● |
| | Scoliosis | ● | ● | ● | ● |
| | Yolk edema | ● | ● | ● | ● |
| | Yolk deformation | ● | ● | ● | ● |
| | Growth retardation | ● | ● | ● | ● |
| | Hatching | | | ● | ● |
| Cardiotoxicity | Pericardial edema | | ● | ● | ● |
| | Heart morphology | | | ● | ● |
| | Heart beating rate (beat/min) | | | | ● |
| Hepatotoxicity | Liver necrosis | | | ● | ● |
| | Yolk resorbtion | | | ● | ● |

Data obtained by FET test revealed that Aristotelia chilensis berry extract was not toxic even at the highest applied dose of 2.5 mg/mL (LC₅₀>2.5 mg/mL) or a minimum of 625 μg/mL delphinidin i.e. a minimum of 875 μg/mL total anthocyanins, where all treated embryos survived up to 120 hpf and developed without any signs of cardiotoxicity, hepatotoxicity, and developmental disorders. In addition, melanocytes of treated embryos are normally developed and pigmented during treatment. These results showed the non-toxicity of Aristotelia chilensis berry extract in high (milligram) doses and suggested potential use in therapy.

### EXAMPLE 2.—ANTIMICROBIAL ACTIVITY

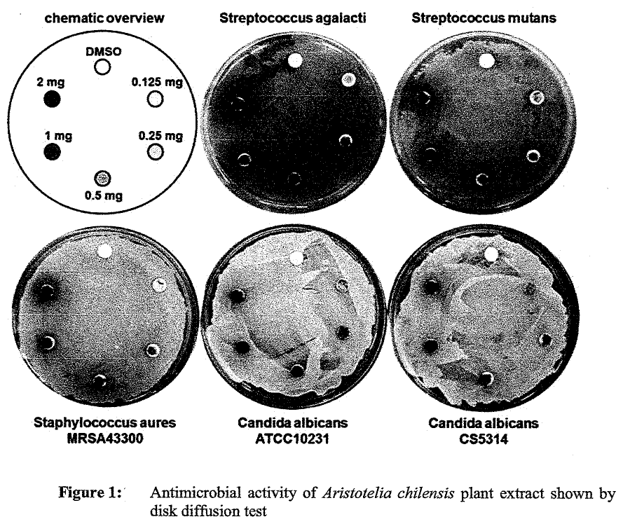

As part of the research, Aristotelia chilensis extract was also tested for antimicrobial activity against various bacterial and fungal pathogens associated with oral and oropharyngeal cavities in humans (Staphylococcus aureus, Streptococcus mutans, Streptococcus agalacti, Streptococcus piogenes and Candida albicans). The activity was tested using a disk diffusion test, and Aristotelia chilensis extract was applied in concentrations of 0.125 mg to 2 mg per disk (minimum 31.25 μg to 500 μg of delphinidin, or 43.75 μg to 700 μg per disc of total anthocyanins). The results shown in FIG. 1 showed neither antibacterial nor antifungal activity against any of the tested strains at the applied doses.

### EXAMPLE 3—ANTIFUNGAL ACTIVITY

As part of a detailed study of the antifungal effect, the minimum inhibitory concentration (MIC) of Aristotelia chilensis extract on three clinical isolates of Candida albicans was determined by the standard microdilution method. The extract was found to have no inhibitory effect on the growth of any of the isolates, even at the highest tested concentration of 10 mg/mL containing a minimum of 2.5 mg/mL of delphinidin and 3.5 mg/mL of total anthocyanins.

In addition to the antimicrobial effect, the effect of Maqui berry extract on the efficacy of clinically approved antifungal drugs (AFL) nystatin, clotrimazole and micafungin was examined using a “checkerboard assay”. The test was performed in RPMI medium without saliva (standard conditions), as well as in the presence of artificial saliva. The tested antifungal concentrations ranged from 0.0312 μg/mL to 2 μg/mL, while the range of tested Maqui berry extract concentrations ranged from 0.0156 mg/mL (3.9 μg/ml delphinidin and 5.46 μg/mL, total anthocyanins) to 4 mg/mL (1 mg/mL delphinidin or 1.4 mg/mL total anthocyanins). The results of the study of the effect of combination therapy (concomitant treatment with drugs and Maqui berry extract) on the growth of clinical isolates of candida showed that the presence of the extract does not affect the efficacy of tested drugs, i.e. did not indicate synergistic or antagonistic effect under standard conditions, either in the presence of artificial saliva, which indicated that the use of Maqui berry extract does not reduce the effectiveness of anti-candida drugs.

### EXAMPLE 4—THE INHIBITION OF CANDIDA FILAMENTATION IN VITRO

Candida albicans is part of the normal oral microflora, where it can cause candidiasis in conditions of weakened immunity, which triggers the morphogenetic transition of yeast into hyphae in a process called filamentation. Filamentation is a major virulence factor in C. albicans, allowing fungi to penetrate epithelium and subepithelial tissues, and colonize internal organs. Therefore, Aristotelia chilensis extract was further tested for inhibition of filamentation in different media reflecting different environmental conditions in which C. albicans is encountered in vivo: sucrose with uridine (UPS+uridine), presence of N-acetyl-glucosamine (GlcNAc) or serum proteins (Spider+FBS). The extract was applied in doses of 50; 200 and 2000 μg/mL corresponding to doses of minimum 12.5; 50 and 500 μg/mL delphinidin, respectively 17.5; 70 and 700 μg/mL total anthocyanins.

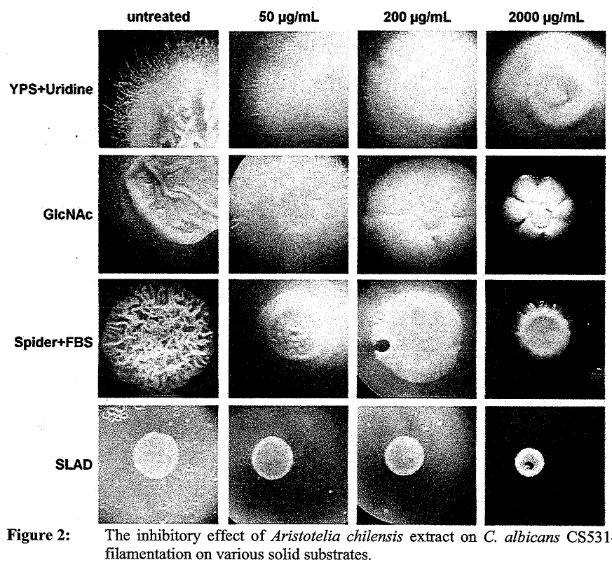

As shown in FIG. 2, Aristotelia chilensis extract inhibited filamentation and changed the morphology of fungal colonies from wrinkled (pathogenic state) to smooth (less pathogenic or non-pathogenic state) on each of the test media, showing that it possesses significant properties in preventing virulence of C. albicans, i.e. under different environmental conditions. In some cases, Aristotelia chilensis extract was active at lower concentrations of 50 μg/mL, while at a concentration of 2 mg/mL it totally inhibited filamentation in all experimental conditions. These results also indicated that Aristotelia chilensis extract contains active ingredients that are likely to affect different signaling pathways that trigger filamentation of C. albicans to various environmental stimuli.

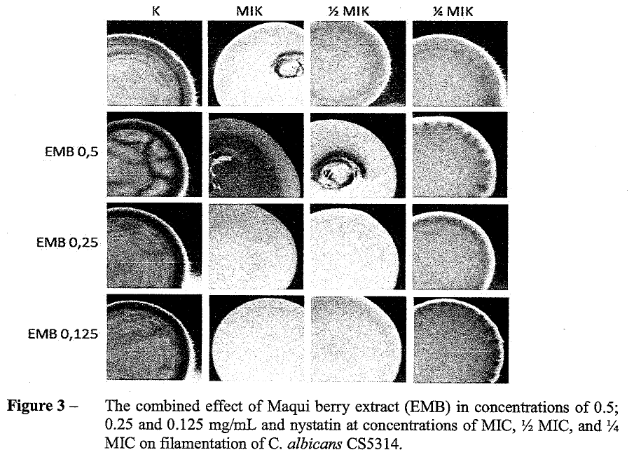

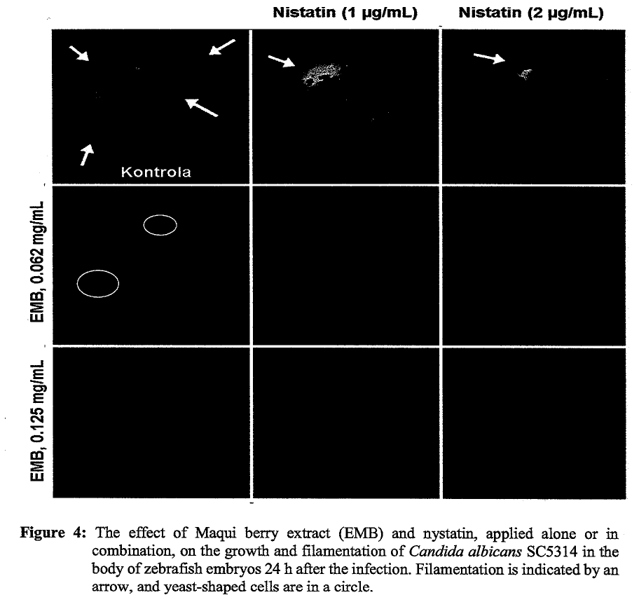

The inhibitory effect of Maqui berry extract and nystatin, one of the most commonly applied topical antifungal drugs, on candida filamentation was investigated in vitro in three clinical isolates in RPMI medium in a checkerboard assay. The extract was tested in a concentration range of 0.125; 0.25 and 0.5 mg/mL (31.25; 62.5 and 125 μg/mL delphinidin, respectively 43.75; 87.5 and 175 μg/mL total anthocyanins), and nystatin in the concentration range 2 μg/mL (MIK), 1 μg/mL (½MIK) and 0.5 μg/mL (¼MIK). The results of this test showed that the filamentation was reduced in the presence of the extract at a concentration of 0.5 mg/mL, while at lower concentrations no inhibitory effect was observed. At the MIK dose of nystatin, the isolates had a reduced growth and no filamentation, while at the ½MIK and ¼MIK doses they grew and filamented. However, the inhibitory effect of combined therapy (Maqui berry extract and nystatin) on candida filamentation was achieved at all tested concentrations of extract and antifungal agents, including doses where individual treatments (extract only or nystatin only) had no effect. In the presence of small doses of extract (0.125 mg/mL and 0.25 mg/mL), the tested strains did not filament even at ¼MIK and ½MIK doses of nystatin, which indicates that in these experimental conditions Maqui berry extract increases the effectiveness of nystatin (FIG. 3).

As, according to literature data, in vitro experimental conditions do not induce oxidative stress in candida, but stimulate filamentation by activating biochemical pathways other than those activated by oxidative stress, the results indicate that the mechanism of performance of Maqui berry extract is not through inhibition of filamentation due to oxidative stress.

### EXAMPLE 5—THE INHIBITION OF CANDIDA FILAMENTATION IN VIVO

The model of the brain infection (hindbrain) of the zebrafish embryo with Candida albicans, described in the literature, is an animal model of the infection system which due to the optical transparency of the embryo body provides the ability to examine and monitor the effect of applied therapeutics on replication, filamentation and fungi dissemination through the body of the zebrafish embryo as a host. The embryos at the developmental stage of 32-34 hpf were injected into the brain through an optic vesicle with 50-75 Candida albicans SC5314 cells, previously labeled with green fluorescent dye. Candida inoculum was prepared in 5% polyvinylpyrrolidone (PVP) solution. Two hours after the microinjection of candida, the infected embryos were treated with Maqui berry extract at a concentration of 0.0625 mg/mL, 0.125 mg/mL and 0.25 mg/mL, as well as nystatin at a concentration of 1 μg/mL (½MIK) and 0.5 μg/mL (¼MIK). Beside individual treatments, the infected embryos were exposed to a combined treatment (nystatin and extract together), in combinations of all mentioned doses. The embryos injected with only 5% PVP represented the control group. After the applied treatments, the zebrafish embryos were reared for the next 4 days at a temperature of 31° C. and monitored daily for survival as well as filamentation and dissemination of candida using fluorescence microscopy.

The results of this in vivo study showed that the extract effectively inhibited candida filamentation in the first 24 h after initiation of treatment, at a concentration of 0.0625 mg/mL. At an extract dose of 0.0625 mg/mL, Candida cells could be seen in small numbers in the brains of infected embryos, and only in the form of yeast. At an extract concentration of 0.125 mg/mL, Candida cells were not detected in most embryos. Since the extract has no antifungal effect, the absence of candida cells could be a consequence of the effect of the extract on the immune system (immunomodulatory effect). These results in treatment with 0.5 μg/mL (¼MIK) and 1 μg/mL (½MIK) dose of nystatin, show that infection (candidiasis) was significantly reduced compared to infection in untreated embryos. However, filamentation was present in both nystatin concentrations. Nevertheless, candida cells were not detected in the body of the infected embryos treated with nystatin and Maqui berry extract, indicating that the combination therapy is very effective in inhibiting filamentation and candida growth, and that Maqui berry extract does not reduce the effect of nystatin in vivo but contributes to complete elimination of the infection.

The results obtained using the zebrafish model showed that Aristotelia chilensis extract is a non-toxic, plant-derived product, and antimicrobial and infectious tests showed a possible use of Aristotelia chilensis plant extract as a new agent against candidiasis. What is important, and is shown by the above experimental results, is that the extract of Aristotelia chilensis is not washed off the mucous membranes immediately after application, so it is evident that its use will allow prolonged exposure of tissue colonized with Candida albicans to active ingredients from the extract.

1. The preparation containing Aristotelia chilensis berry extract and/or extracts of other berries rich in anthocyanins as an active substance, for use as a prophylactic or therapeutic agent for treating fungal infections by inhibiting filamentation.

2. The preparation according to claim 1, wherein, the fungal infection is caused by fungi from the genus Candida, especially Candida Albicans.

3. The preparation, according to claim 1, wherein, it can be used for treating fungal infections of the urogenital, digestive tract, on skin and mucous membranes, as well as for treating systemic diseases.

4. The preparation, according to claim 1, wherein, with the addition of acceptable substances, it can be used in any pharmaceutically acceptable form including, but not limiting to powder, lozenge, gel or solution.

5. The preparation, according to claim 1 4, wherein, it can be used as a medicine, dietary supplement or a supplement to cosmetic products.

6. The preparation according to claim 1, wherein, it can be used alone or in combination with other preparation for treating and prophylaxis of fungal infection.
